(19) United States
(12) Patent Application Publication
(10) Pub. No.: US 2017/0049675 A1
(43) Pub. Date: Feb. 23, 2017

(54) PHARMACEUTICAL COMPOSITION AND METHOD FOR TREATMENT AND PREVENT OF HAIR LOSS.

(71) Applicant: Thierry Momplaisir, Villanova, PA (US)

(72) Inventor: Thierry Momplaisir, Villanova, PA (US)

(21) Appl. No.: 15/228,119

(22) Filed: Aug. 4, 2016

Related U.S. Application Data
(60) Provisional application No. 62/208,714, filed on Aug. 23, 2015.

(51) Int. Cl. A61K 8/49; A61Q 7/00; A61K 8/06; A61K 8/92

(52) U.S. Cl. CPC A61K 8/4953; A61K 8/922; A61K 8/4946; A61Q 7/00; A61K 8/06; A61K 2800/74; A61K 2800/524

(57) ABSTRACT

A pharmaceutical composition and method for treating and preventing hair loss due to multiple factors are disclosed. The composition may contain a natural oil forming an oil base, a first quantity of minoxidil and a second quantity of an antifungal agent, such that the minoxidil and the antifungal agents are equally distributed throughout the oil base. The pharmaceutical composition further includes an excipient solution made up of benzoic acid, cetyl alcohol, isopropyl myristate, polysorbate 60, potassium hydroxide, propylene glycol, purified water and stearyl alcohol.

### CROSS REFERENCES TO RELATED APPLICATIONS

This application claims priority from U.S. Provisional Patent Application No. 62/208,714 filed on Aug. 23, 2015,which application is herein incorporated by reference as if set forth in its entirety.

### FIELD OF THE INVENTION

Embodiments of the present disclosure relate generally to male and female pattern baldness and more specifically to a method for preparing a pharmaceutical composition for treatment and prevention of hair loss caused by multiple factors.

### BACKGROUND OF THE INVENTION

Hair loss treatment is quite challenging and expensive with often-limited results. Androgenic alopecia (also known as male and female pattern baldness) is hereditary in both men and women due to genetic as well as environmental factors. Conversion of elevated levels of testosterone to dihydrotestosterone (DHT) might be one of the reasons for destroying hair follicles resulting in hair loss. The male and female pattern baldness is triggered by additional factors comprising autoimmune disorder (lupus), drugs, particularly chemotherapy and physical stress. Further, hair loss may be caused by fungal infection on the scalp. The fungal infection might occur anywhere on the body including scalp causing patches of hair loss resulting in baldness. For example, Tinea Capitis is a fungal infection that occurs on the scalp. The infection usually begins as a small pimple that progressively expands in size, leaving scaly patches of temporary baldness. The fungus gets into hair fibers in the affected area and makes the hair follicles brittle in nature that easily break off, leaving a bald patch of skin. Affected areas are often itchy, red, and inflamed, with scaly patches that may blister and ooze.

The conventional drugs work effectively either for treating hair loss caused by genetic factors or by environmental factors but are ineffective against multiple factors. For example, drug used for treating or preventing hair loss due to genetic factors may not be effective in treating hair loss due to environmental factors or due to fungal infection. Hence, there is a need for an effective pharmaceutical composition for treatment and prevention of male and female pattern baldness caused by multiple factors.

### SUMMARY

Various embodiments provide a pharmaceutical composition and method for treatment and prevention of hair loss.

According to one embodiment of the present disclosure, a pharmaceutical composition for treatment of hair loss comprising a natural oil forming an oil base, a first quantity of minoxidil and a second quantity of an antifungal agent, wherein the minoxidil and the antifungal agent are equally distributed throughout the oil base. The pharmaceutical composition further comprises an excipient solution wherein the natural oil and the excipient solution hold the minoxidil and the antifungal agent in equal concentrations throughout the pharmaceutical composition. The excipient solution comprises benzoic acid, cetyl alcohol, isopropyl myristate, polysorbate 60, potassium hydroxide, propylene glycol, purified water and stearyl alcohol. In an embodiment, the antifungal agent comprises at least one of ketoconazole and miconazole nitrate whereas the natural oil comprise oil extracted from at least one of argan, olive, pomegranate seed, rosemary, avocado, jojoba and coconut.

According to another embodiment of the present disclosure, a method of preparing the pharmaceutical composition for treatment of hair loss comprising adding first quantity of minoxidil solution to natural oil forming an emulsion, mixing a second quantity of an antifungal agent to the emulsion forming an active suspension, homogenizing the active suspension by an excipient solution and stabilizing the homogenized solution for at least 5-6 hours at room temperature, wherein the natural oil and the excipient solution hold the minoxidil solution and the antifungal agent at equal concentrations throughout the composition to produce equal pharmacological effect per unit. In an embodiment, the first quantity of minoxidil make up to at least one of 5% per unit and 2% per unit whereas the second quantity of the antifungal agent makeup to 2% per unit of the pharmaceutical composition.

Several embodiments are described below, with reference to diagrams for illustration. It should be understood that numerous specific details are set forth to provide a full understanding of the invention. One skilled in the relevant art, however, will readily recognize that the invention can be practiced without one or more of the specific details, or with other methods, etc. In other instances, well-known structures or operations are not shown in detail to avoid obscuring the features of the invention.

### BRIEF DESCRIPTION OF DRAWINGS

The teachings of the present invention can be readily understood by considering the following detailed description in conjunction with the accompanying drawings, in which:

FIG. 1 depicts a block diagram illustrating a pharmaceutical composition for treatment and prevention of male and female pattern baldness caused by multiple factors in one embodiment of the present disclosure;

FIG. 2A though FIG. 2C depict the diagrams illustrating active components of the pharmaceutical composition in an embodiment of the present disclosure;

FIG. 3A through FIG. 3H depict the diagrams illustrating inactive components of the pharmaceutical composition in an embodiment of the present disclosure; and

FIG. 4 shows a block diagram illustrating a method of preparation of the pharmaceutical composition in one embodiment of the present disclosure.

### DETAILED DESCRIPTION

Various embodiments provide a pharmaceutical composition and method for treating and preventing hair loss due to multiple factors.

The illustrative composition and method embodiments described herein are not meant to be limiting. It may be readily understood that certain aspects of the disclosed composition and method can be arranged and combined in a variety of different configurations, all of which are contemplated herein.

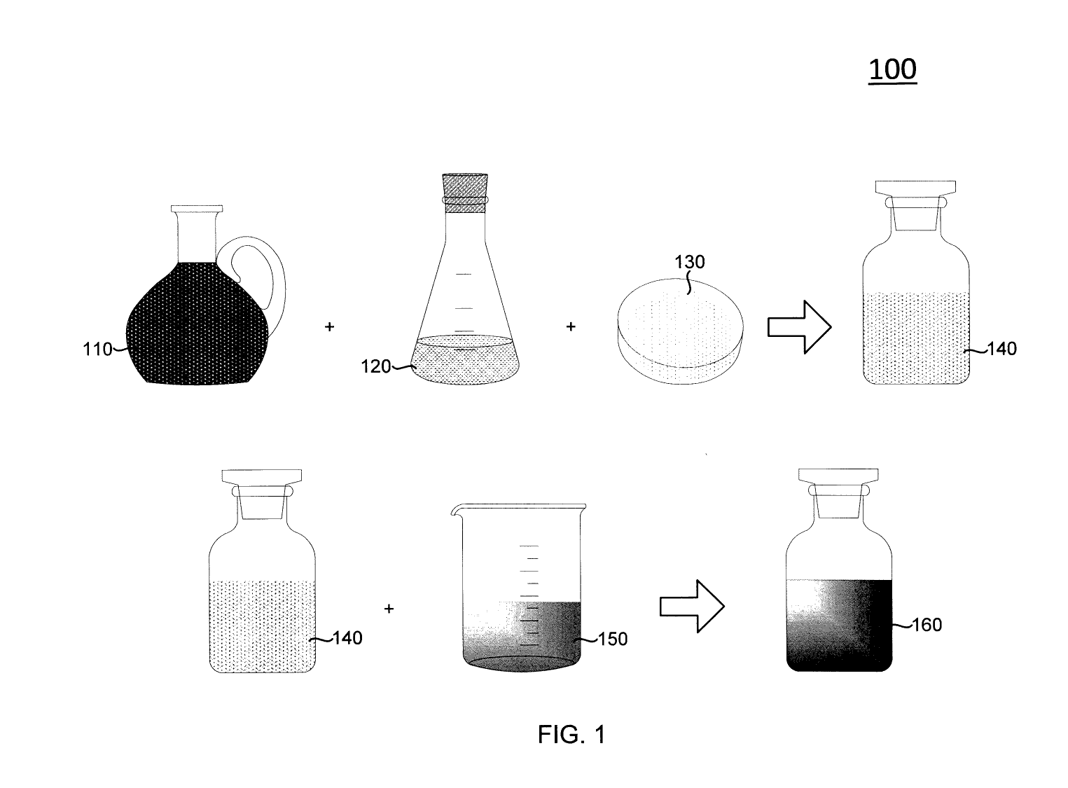

FIG. 1 depicts a block diagram illustrating a pharmaceutical composition for treatment and prevention of male and female pattern baldness caused by multiple factors in one embodiment of the present disclosure. The pharmaceutical composition for treating hair loss caused by multiple factors comprises natural oil 110, minoxidil solution 120, an antifungal agent/component 130 and additional ingredients 140. In one embodiment, an active suspension 140 is prepared by mixing the natural oil 110 (for example at least one of coconut oil, argan oil, olive oil, pomegranate seed oil, rosemary oil, avocado oil and jojoba oil) with at least one of 5% or 2% minoxidil solution 120 along with the antifungal component 130 (say at least one of ketoconazole or miconazole). The natural oil 110 forms an oil base for the minoxidil 120 and the antifungal agent 130 which prevents dry scalp and promotes hair growth.

Besides to this active suspension 140, an excipient solution 150 is prepared by using benzoic acid, cetyl alcohol, isopropyl myristate, polysorbate 60, potassium hydroxide, propylene glycol, purified water and stearyl alcohol. The active suspension 140 is then mixed with the excipient solution 150 and stabilized for at least 5 to 6 hours to obtain a stabilized homogeneous composition 160.

The natural oil 110 and excipient solution 150 hold the minoxidil solution and antifungal component together in such a way that both the minoxidil and antifungal component are equally distributed throughout the composition concentrations of the minoxidil 120 and antifungal component 130 remains same throughout the stabilized composition 160. As the final stabilized composition 160 comprises both minoxidil 120 and antifungal component 130 that are distributed at equal concentrations, the stabilized composition 160 is effective in treating or preventing hair loss caused by multiple factors for example, both genetic and environmental factors.

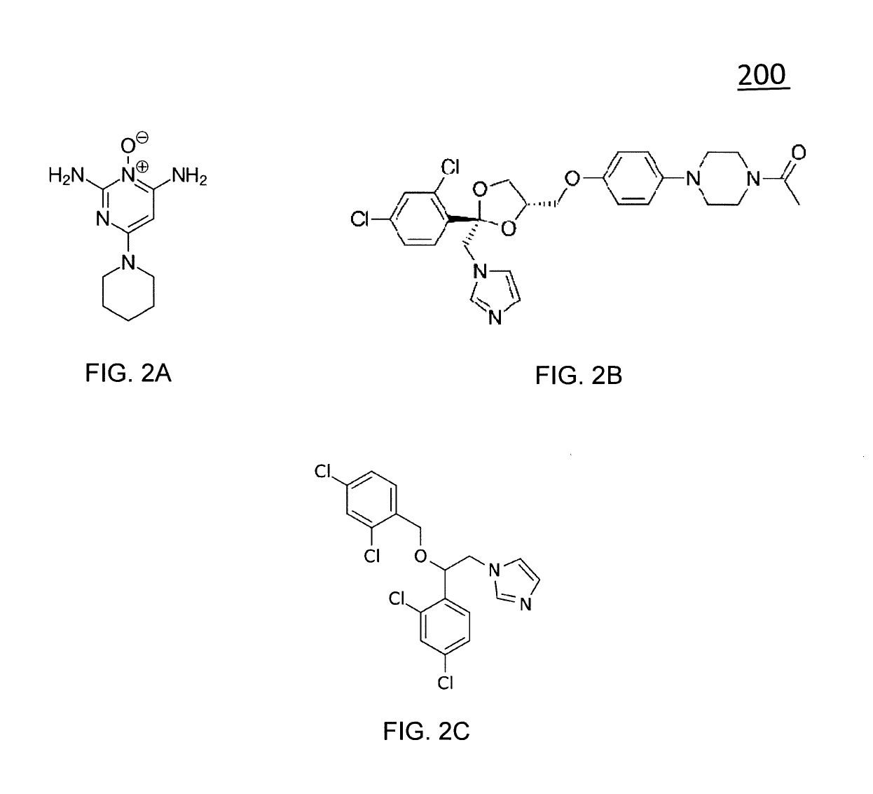

FIG. 2A though FIG. 2C depict the diagrams illustrating active components of the 25 pharmaceutical composition in an embodiment of the present disclosure. The pharmaceutical composition comprises two categories of components namely active components and inactive components. The active components of the pharmaceutical composition comprise natural oil, minoxidil (FIG. 2A) and at least one of ketoconazole (FIG. 2B) or micronosole nitrate (Miconazole) (FIG. 2C).

The active components directly involve in the treatment and prevention of male and female pattern baldness. The minoxidil (FIG. 2A) is an active component that increases hair follicles by increasing flow rate of blood in blood vessels and further thickens the shaft of existing hair so that it grows in thicker. In an embodiment, 5% minoxidil solution is used for male pattern baldness whereas 2% minoxidil solution is used for female pattern baldness. The ketoconazole (FIG. 2B) and miconazole nitrate (FIG. 2C) are the antifungal agents that inhibit fungal growth on the scalp. Further ketoconazole or miconazole nitrate or both in combination lowers the testosterone levels to a limited extent that helps in reducing the hair loss.

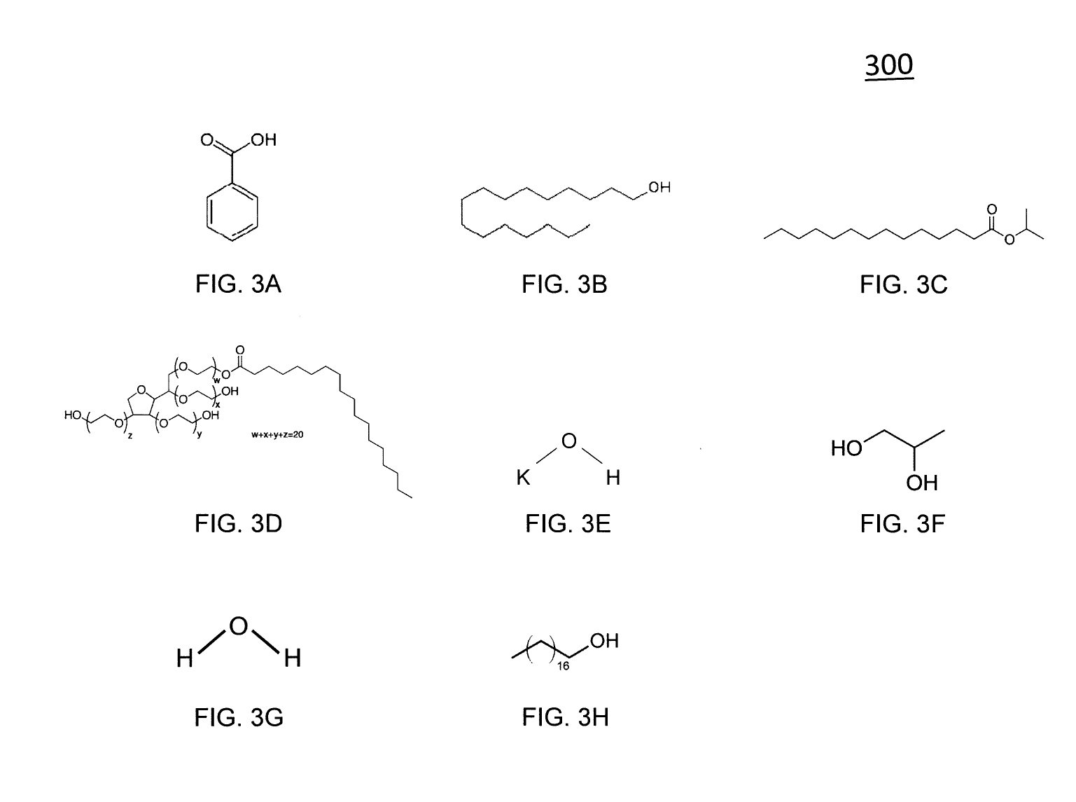

FIG. 3A through FIG. 3H depict the diagrams illustrating inactive components of the pharmaceutical composition in an embodiment of the present disclosure. The inactive components of the pharmaceutical composition are pharmaceutically acceptable excipients comprising benzoic acid (FIG. 3A), cetyl alcohol (FIG. 3B), isopropyl myristate (FIG. 3C), polysorbate 60 (FIG. 3D), potassium hydroxide (FIG. 3E), propylene glycol (FIG. 3F), purified water (FIG. 3G) and stearyl alcohol (FIG. 3H).

The inactive components have no direct involvement in the treatment or prevention of hair loss. However, the inactive components FIG. 3A through FIG. 3H) along with a suitable amount of natural oil 110 holds the active components (FIG. 2A through FIG. 2C) together at equal concentrations throughout the composition 160. Further varying proportions of the inactive components results in various forms of the composition such as solutions, gels, suspensions, and the like used for topical application.

Liquids tend to be immiscible when force of attraction between molecules of a same liquid is greater than force of attraction between molecules of two different liquids. This immiscible property of liquids poses difficulty in processing and preparation of a pharmaceutical composition comprising different liquids or active components to treat male and female pattern baldness caused by multiple factors. Hence an effective method of preparing a pharmaceutical composition comprising equally distributed concentrations of minoxidil and at least one of the ketoconazole or the miconazole, benzoic acid, cetyl alcohol, isopropyl myristate, polysorbate 60, potassium hydroxide, propylene glycol, purified water and stearyl alcohol for treatment and prevention of baldness is further described below.

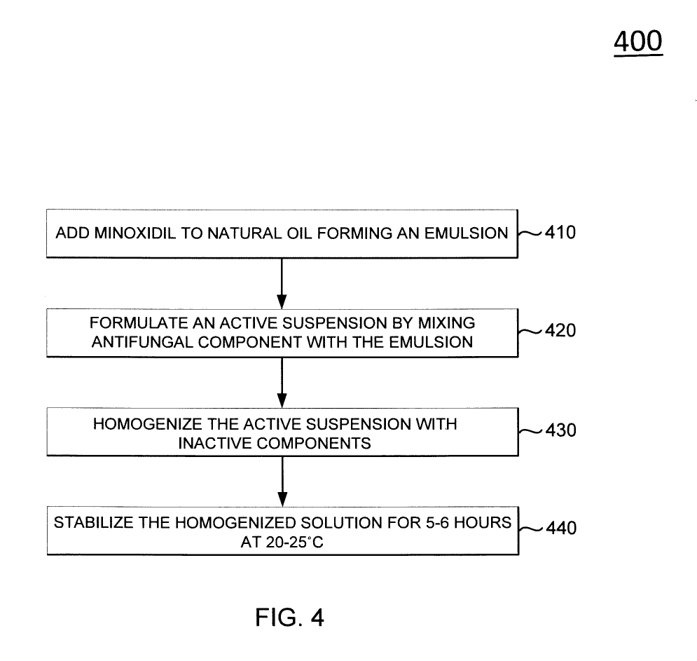

FIG. 4 shows a block diagram illustrating a method of preparation of the pharmaceutical composition in one embodiment of the present disclosure.

In block 410, an emulsion of the natural oil 110 and the minoxidil 120 is prepared by adding suitable amount of the minoxidil 120 to the natural oil 110 at room temperature. In an embodiment, 2% minoxidil solution is used for female pattern baldness whereas 5% minoxidil solution is used for male pattern baldness.

In block 420, the emulsion obtained from block 410 is mixed with an antifungal component comprising at least one of ketoconazole or miconazole nitrate resulting in an active suspension 140. In one embodiment, the active suspension 140 is prepared by using 2% (100 mg/application) of the antifungal component.

In block 430, the active suspension 140 is mixed with the inactive components and homogenized to form a uniform solution. In an embodiment, the inactive components comprise benzoic acid, cetyl alcohol, isopropyl myristate, polysorbate 60, potassium hydroxide, propylene glycol, purified water and stearyl alcohol.

In block 440, the homogenized solution obtained from block 430 is stabilized by leaving the solution for at least 5-6 hours at room temperature. The stabilization time may be optimized based on equipment and manufacturing process. In the stabilization process, a chemical reaction may occur to stabilize and disperse the minoxidil solution and the antifungal component uniformly at equal concentrations throughout the composition 160. Thus equally distributed minoxidil and antifungal component of the stabilized solution is pharmacologically effective in treating male and female pattern baldness caused by multiple factors.

The stabilized solution is further stored in a cool and dry place at room temperature. This solution is topically applied on the scalp to treat male and female pattern baldness caused by multiple factors. In an embodiment, this solution may be utilized in preparation of various gels, foams and other means of topical application for treating hair loss.

In yet another embodiment, the stabilized solution (composition 160) obtained from block 440 comprises at least 85% natural oil, 5% minoxidil solution, 2% antifungal component and 8% inactive components for treating male pattern baldness. In contrast, the composition for treating female pattern baldness comprises 88% natural oil, 2% minoxidil solution, 2% antifungal component and 8% inactive components.

Thus, the pharmaceutical composition of the present disclosure comprising more than one active component is used as a hair loss preventive solution for treating male and female pattern baldness caused by multiple factors.

While various embodiments of the present disclosure have been described above, it should be understood that they have been presented by way of example only, and not limitation. Thus, the breadth and scope of the present disclosure should not be limited by any of the above-discussed embodiments, but should be defined only in accordance with the following claims and their equivalents.

1. A pharmaceutical composition for treatment of hair loss comprising:
a natural oil forming an oil base;
a first quantity of minoxidil; and
a second quantity of an antifungal agent,
wherein the minoxidil and the antifungal agent are equally distributed throughout the oil base.

2. The pharmaceutical composition of claim 1, wherein the antifungal agent comprises at least one of ketoconazole and miconazole nitrate.

3. The pharmaceutical composition of claim 2, wherein the natural oil comprises oil extracted from at least one of argan, olive, pomegranate seed, rosemary, avocado, jojoba and coconut.

4. The pharmaceutical composition of claim 3, further comprising an excipient solution wherein the excipient solution comprises benzoic acid, cetyl alcohol, isopropyl myristate, polysorbate 60, potassium hydroxide, propylene glycol, purified water and stearyl alcohol.

5. The pharmaceutical composition of claim 4, wherein the natural oil and the excipient solution hold the minoxidil and the antifungal agent in equal concentrations throughout the pharmaceutical composition.

6. The pharmaceutical composition of claim 5, wherein the first quantity make up to 5% per unit and second quantity make up to 2% per unit of the pharmaceutical composition.

7. The pharmaceutical composition of claim 5, wherein the first quantity make up to 2% per unit and second quantity make up to 2% per unit of the pharmaceutical composition.

8. The pharmaceutical composition of claim 5, wherein volumetric percentage of the natural oil and the excipient solution are in the ratio of 10:1 per unit of the pharmaceutical composition.

9. A method of preparing a pharmaceutical composition for treatment of hair loss comprising:
adding a first quantity of minoxidil solution to natural oil thereby forming an emulsion;
mixing a second quantity of an antifungal agent to the emulsion forming an active suspension;
homogenizing the active suspension by an excipient solution; and
stabilizing the homogenized solution for a specific time period at room temperature,
wherein the natural oil and the excipient solution hold the minoxidil solution and the antifungal agent at equal concentrations throughout the composition to produce equal pharmacological effect per unit.

10. The method of claim 9, wherein the antifungal agent comprises at least one of ketoconazole and the miconazole.

11. The method of claim 10, wherein the natural oil comprises oil extracts from at least one of argan, olive, pomegranate seed, rosemary, avocado, jojoba and coconut.

12. The method of claim 11, wherein the excipient solution comprises benzoic acid, acetyl alcohol, isopropyl myristate, polysorbate 60, potassium hydroxide, propylene glycol, purified water and stearyl alcohol.

13. The method of claim 12, wherein the first quantity make up to 5% per unit and second quantity make up to 2% per unit of the pharmaceutical composition.

14. The method of claim 12, wherein the wherein the first quantity make up to 2% per unit and second quantity make up to 2% per unit of the pharmaceutical composition.

15. A hair loss preventive solution comprising:
a natural oil forming an oil base, wherein the natural oil comprise oil extracted from at least one of argan, olive, pomegranate seed, rosemary, avocado, jojoba, and coconut;
a first quantity of minoxidil wherein first quantity make up to at least one of 5% per unit and 2% per unit of the hair loss preventive solution;
a second quantity of an antifungal agent comprising at least one of ketoconazole and miconazole, wherein the second quantity makeup to 2% per unit of the hair loss preventive solution; and
an excipient solution comprising benzoic acid, acetyl alcohol, isopropyl myristate, polysorbate 60, potassium hydroxide, propylene glycol, purified water and stearyl alcohol,
wherein the natural oil and the excipient solution hold the minoxidil and the antifungal agent in equal concentrations throughout the hair loss preventive solution.
